(19) United States
(12) Patent Application Publication
(10) Pub. No.: US 2025/0069516 A1
(43) Pub. Date: Feb. 27, 2025

(54) APPARATUS AND METHOD FOR ENABLING PERSONALIZED COMMUNITY POST-STROKE REHABILITATION

(71) Applicant: Versitech Limited, Hong Kong (HK)

(72) Inventors: Wai Tung Wilton FOK, Hong Kong (HK); Kui Kai Gary LAU, Hong Kong (HK); Yingxian CHEN, Hong Kong (HK); Hau Lam Elton YEUNG, Hong Kong (HK); Wun Yin LAM, Hong Kong (HK)

(21) Appl. No.: 18/813,083

(22) Filed: Aug. 23, 2024

Related U.S. Application Data
(60) Provisional application No. 63/578,370, filed on Aug. 24, 2023.

(51) Int. Cl. G09B 5/06; A61B 5/00; A61B 5/11

(52) U.S. Cl. CPC G09B 5/065; A61B 5/0022; A61B 5/1118; A61B 5/1128; A61B 5/486; A61B 5/7465

(57) ABSTRACT

A system for providing personalized community-based post-stroke rehabilitation is disclosed. The system includes a user-end module, a cloud platform module, and a therapist-end module. The user-end module is configured to provide schedule information with instructions of at least one specific exercise and to show at least one image or video, in which the user-end module provides a camera view page to record a target image or video and to record a target user's performance metric. The cloud platform module is configured to receive and store the target user's performance metric from the user-end module. The therapist-end module is permitted to log in the cloud platform module and receive the target user's performance metric from the cloud platform module, and the therapist-end module is further configured to visualize the target user's performance metric so as to show exercise waveform comprising quantitative data for qualitative analysis.

### TECHNICAL FIELD

The present invention generally relates to validation of digital human activity evaluation. More specifically, the present invention relates to a method and an apparatus for validation of consumer-grade digital camera-based human activity evaluation for upper limb exercises and for development of a therapist-guided, automated telerehabilitation framework and platform for stroke rehabilitation.

### BACKGROUND

Stroke is a global health issue, causing significant disability and death. As the prevalence of stroke increases, there is a pressing need for innovative rehabilitation solutions to address the growing demand and mitigate the impact on public health systems. These challenges arise from three main aspects as stated below.

### I: Global Burden of Stroke and Demand for Rehabilitation Services

Stroke is a leading cause of disability and death worldwide. Timely and adequate rehabilitation is critical in facilitating stroke recovery, ultimately impacting one's quality of life. With an aging population and an increasing prevalence of non-communicable diseases, including stroke, there has been a high and rising demand for rehabilitation services globally. During the coronavirus-disease 2019 (COVID-19) pandemic, face-to-face rehabilitation services have been significantly impacted globally, further limiting stroke survivors' recovery potential. The World Health Organization concomitantly declared enhancing rehabilitation services as one of the urgent priorities; innovative solutions are urgently required to improve access to stroke rehabilitation services and reduce the public healthcare burden.

### II: Current Strategies to Facilitate Motor Rehabilitation in the Community Setting

Other than conventional in-person rehabilitation services. The COVID-19 pandemic has shown a significant increase in real-world adoption of telemedicine and telerehabilitation globally, as seen in various specialties, including cardiology, respiratory medicine, neurology, and orthopedics. These platforms allow rehabilitation services to be continued despite physical, functional, and financial barriers, including costs and difficulty traveling to and from in-person service centers, perception of added stress for carers, and lack of incentives or motivations.

Current telerehabilitation solutions rely heavily on one's subjective reporting through video conferencing or phone interviews, which can be labor intensive and subject to recall biases. Other technological solutions utilize sensor-based (e.g., Xsens and smartwatches) and camera-based (e.g., Vicon and Kinect) devices for obtaining objective measurements, and have demonstrated excellent prediction and measurement of one's exercise performance, namely the range of motion for home-based motor rehabilitation. Indeed, in addition to conventional therapy, these systems have shown benefits in improving the functional outcome of patients with stroke. However, these approaches fall short in real-world applications for home-based telerehabilitation, given their high costs per unit and space requirements.

### III: Advanced Technology for Facilitating Home-Based Motor Rehabilitation

Recent advancements in artificial intelligence, sensor technology, and hardware specifications allowed people to tap into the potential of consumer-grade products (i.e., smartphones and tablet devices) as an affordable and accessible alternative. Human pose estimation is a state-of-the-art computer vision approach for segmenting and obtaining key information about one's body joints in space, including shoulders, elbows, knees, and more, as shown in FIG. 1.

The illustration of FIG. 1 highlights the key joints of the body frame predicted using the computer vision algorithm. Key joints of interest include the head, shoulders, elbows, wrists, hips, knees, and ankle joints. A sample frontal view of the movement waveform of participant performing left shoulder abduction, predicted by different pose estimation models, is shown on the right. This view is constructed with all 13 key joints plotted together.

According to the illustration, it is a process of identifying and predicting the position and orientation of human body parts using an image or video captured via a sensor (e.g., Red Green Blue (RGB) digital camera, depth camera, infrared sensors). The human pose estimation model initially relies on high computational power devices to run the complex and convoluted algorithm for optimal performance. Today, more sophisticated and compressed models exist and can achieve similar performance on a wide range of consumer-grade devices.

Some devices have achieved camera-based motor-related rehabilitation. However, these devices require a console to power their pose estimation models on the environment captured by the built-in infrared camera. Additionally, they need a relatively large spatial area to adequately capture the target user and surrounding environment. These factors altogether render this approach challenging to scale up and implement for home-based exercise, particularly in low- and middle-income countries (LMICs).

### SUMMARY OF INVENTION

It is an objective of the present invention to provide a method and apparatus for smartphones and artificial intelligence to facilitate personalized-community post-stroke rehabilitation. Timely and adequate rehabilitation is critical in facilitating post-stroke recovery. However, the organization and delivery of rehabilitation are resource-demanding and are only available to approximately 25% of stroke survivors in low-to-middle-income countries. Improving access to stroke rehabilitation services through innovative solutions is therefore urgently required. Telerehabilitation, which transitions care to home and community settings, has emerged as a promising solution. However, current approaches using video tutorials, teleconferences, or other specialized devices face inherent shortcomings that limit their uptake.

The present disclosure proposes and validates the use of an open-source, markerless motion capture model with consumer-grade devices to overcome these challenges. The solution enables reliable measurement of the end range of motion during upper limb exercises with near-perfect waveform similarity and intraclass correlation to that of the gold standard Kinect approach. An automated telerehabilitation framework incorporating the validated markerless technique is developed to facilitate a seamless telerehabilitation process. It enables personalized rehabilitation plans with real-time feedback and individual progress reports using objective quantitative and qualitative features to improve patient monitoring and management, as well as home-based rehabilitation service uptake and compliance. Further incorporation of training modalities (e.g., cognitive and speech) and other Internet of Things (IoT) devices (e.g., biometric sensors for monitoring vitals) may facilitate a holistic telerehabilitation process. The present disclosure serves as a proof-of-concept in preparation for the future development of a detailed model of care, and feasibility, usability, and cost-effectiveness studies of an automated telerehabilitation platform and framework in improving post-stroke rehabilitation and functional outcomes.

To overcome these aforementioned challenges, a telerehabilitation platform accessible via smartphones or tablets is proposed in the present disclosure. This cutting-edge solution enables users to participate in personalized rehabilitation programs at home, offering customized instructions, real-time automated feedback, and progress reports to effectively improve patient outcomes.

FIG. 2 depicts the development of information and communication systems using systems such as 5G IoT and cloud services. With these systems, “mobile health” or “mHealth” rapidly grows as a field involving the use of mobile devices such as smartphones, tablets, and wearable devices in healthcare delivery and management. To address hardware limitations and allow accessibility and scalability for facilitating rehabilitation in the community, the built-in RGB camera in smartphone devices is utilized. Computer vision models are able to predict the human body and key point locations in space (e.g., x and y coordinates of the elbow joint in space at time t), and this information is valuable for evaluating one's rehabilitation performance. The performance is then objectively and automatically evaluated with the inference of the joint angles, velocity, smoothness of movement, and other properties using the body frame key points. Automating the evaluation process and incorporating gamification elements in the feedback system resemble an on-demand “smart therapist” while facilitating home-based rehabilitation.

By developing a smartphone model, the aim is to minimize the physical barrier carried by the conventional in-person services provided at rehabilitation day centers. The smartphone-based rehabilitation evaluation allows stroke survivors to initiate their training sessions at any time and any place, fitting the rehabilitation session to one's schedule with great flexibility. Therefore, a platform named SmartRehab system is designed, which acts as an AI therapist for facilitating quality home-based rehabilitation exercises through utilizing computer vision models, including image processing, object detection, and pose estimation for analyzing and evaluating one's performance. The model captures the image of the patient, recognizes the key points of the patient's body frame, and analyzes the performance using biometric data and joint angles. During the exercise, SmartRehab system provides real-time visual and audio feedback to correct any inaccuracies in the exercise, simulating a therapist providing comments during in-person rehabilitation sessions, using metrics and rubrics developed by the experts from the Hong Kong Society for Rehabilitation (HKSR) and HKU Stroke. In addition, a sensor-based earphone is connected during rehabilitation, enabling direct auditory instruction and feedback. Its potential role includes respiratory rate and pulse oximeter functions in stroke, cardiac, and respiratory patient populations for telerehabilitation.

Using a cloud-based approach, rehabilitation data can be uploaded and stored. Responsible therapists can then remotely access and review the patient's progression, and even prescribe and modify rehabilitation exercise plans to facilitate personalized therapy. The cloud-based system and automated evaluation using an AI model allow for remote and telerehabilitation in the community, significantly alleviating stress for therapists and reducing wait times for stroke survivors.

In summary, the SmartRehab system can be delivered as an application on a smartphone and smart device platform, making it accessible and scalable for community use. It provides a unique and innovative solution for stroke patients in need of rehabilitation, especially during the COVID-19 era, when rehabilitation services have been severely affected globally.

In accordance with a first aspect of the present invention, a system for providing personalized community-based post-stroke rehabilitation is disclosed. The system includes a user-end module, a cloud platform module, and a therapist-end module. The user-end module is configured to provide schedule information with instructions of at least one specific exercise and to show at least one image or video, in which the user-end module provides a camera view page to record a target image or video and to record a target user's performance metric. The user-end module comprises a feeding module, a human activity recognition module, and a human activity evaluation module. The feeding module is configured to acquire the image or video from a consumer-grade smartphone or tablet device. The human activity recognition module is configured to receive the image or video from the feeding module and to leverages a human pose estimation model to analyze input data. The human activity evaluation module is configured to receive results transmitted from the human activity recognition module and to evaluate performance of the results based on performance metric information which is rule-based or template-based. The cloud platform module is configured to receive and store the target user's performance metric from the user-end module. The therapist-end module is in communication with the user-end module via the cloud platform module. The therapist-end module is permitted to log in the cloud platform module and receive the target user's performance metric from the cloud platform module, and the therapist-end module is further configured to visualize the target user's performance metric so as to show exercise waveform comprising quantitative data for qualitative analysis.

In accordance with a second aspect of the present invention, a method for providing personalized community-based post-stroke rehabilitation is disclosed. The method includes steps as follows: providing, by a user-end module, schedule information with instructions of at least one specific exercise; showing, by the user-end module, at least one image or video, wherein the user-end module provides a camera view page to record a target image or video and to record a target user's performance metric. The user-end module comprises: a feeding module configured to acquire the image or video from a consumer-grade smartphone or tablet device; a human activity recognition module configured to receive the image or video from the feeding module and to leverages a human pose estimation model to analyze input data; and a human activity evaluation module configured to receive results transmitted from the human activity recognition module and to evaluate performance of the results based on performance metric information which is rule-based or template-based. The method further includes steps as follows: receiving and storing, by a cloud platform module, the target user's performance metric from the user-end module; logging, by using a therapist-end module, in the cloud platform module to receive the target user's performance metric from the cloud platform module; and visualizing, by using the therapist-end module, the target user's performance metric so as to show exercise waveform comprising quantitative data for qualitative analysis.

With the above configuration, this study proposes a more accessible and affordable solution using an improved pose estimation model that relies solely on consumer-grade devices in combination with human activity evaluation methods to automate post-stroke gross motor exercise evaluation. The proposed solution is validated against the existing and widely accepted Microsoft Kinect pose estimation model, and a telerehabilitation model of care is developed using the validated automated evaluation method to lay the foundation for future trials to determine the efficacy of such platforms.

### BRIEF DESCRIPTION OF DRAWINGS

Embodiments of the invention are described in more details hereinafter with reference to the drawings, in which:

FIG. 1 depicts key joints of the body frame predicted using the computer vision algorithm;

FIG. 2 depicts the development of information and communication systems using systems such as 5G IoT and cloud services;

FIG. 3 depicts a schematic drawing of an approach to data collection in some embodiments of the present invention;

FIG. 4 depicts a schematic diagram outlining the framework of a system designed to facilitate telerehabilitation according to some embodiments of the present invention;

FIG. 5 depicts user interface of the mobile application for the system of FIG. 4 according to some embodiments of the present invention;

FIG. 6 depicts a schematic diagram for showing a therapist's portal system of the SmartRehab system of FIG. 4 according to some embodiments of the present invention;

FIG. 7A and FIG. 7B show graphs for visualization of movement waveforms captured during shoulder abduction and elbow extension; and

FIG. 8 depicts a schematic architecture of a system for a personalized community-based post-stroke rehabilitation according to one embodiment of the present invention.

### DETAILED DESCRIPTION OF THE INVENTION

In the following description, systems and method for enabling personalized community post-stroke rehabilitation and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The preparation for the work of the present invention and the related work are described first as follows.

### (1) Data Collection

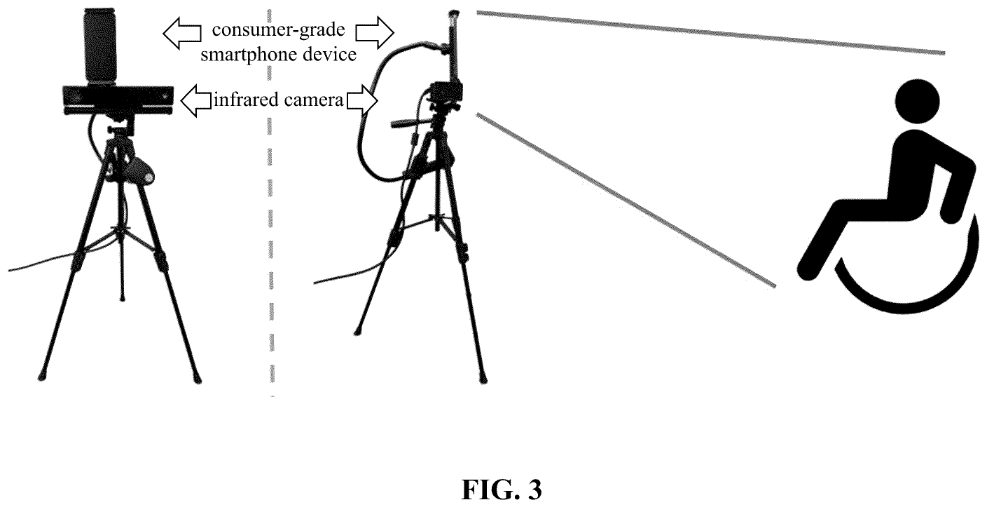

FIG. 3 depicts a schematic drawing of an approach to data collection in some embodiments of the present invention. Video recordings of participants performing the thirteen listed rehabilitation exercises, as shown in Table 1 below, are made using a consumer-grade smartphone and an infrared camera setup, under a well-lit environment. FIG. 3 illustrates the front view (left) and side view (right) of the recording setup. The setup comprises a consumer-grade smartphone device stacked over the infrared camera, elevated 1 m above ground level using a tripod stand.

The stacked devices are aligned and calibrated using the built-in levelling tool, with the consumer-grade smartphone inverted to minimize the distance between the RGB digital camera and the infrared camera. The devices are supported by a tripod stand 1 m above the ground to minimize tilting and video distortion. Each recording session involves the participant performing ten repetitions of all listed rehabilitation exercises to the best of their ability. Video recordings on the consumer-grade smartphone device and the infrared camera are captured at 60 frames per second (fps).

TABLE 1
List of post-stroke gross motor exercises

| Exercises in SmartRehab | Domain |
|---|---|
| Shoulder abduction (Unilateral/Bilateral) ^{#} | Upper Limb |
| Shoulder flexion | Upper Limb |
| (Unilateral/Bilateral/Assisted) ^{#} | |
| Elbow extension (Unilateral/Assisted) ^{#} | Upper Limb |
| Shoulder external rotation | Upper Limb |
| Single leg stance | Lower Limb |
| Pre-gait swing | Lower Limb |
| Lateral flexion and elongation of the trunk | Trunk |
| Weight shift (Sitting/Standing) | Balance & Core |

^{#} Included and validated in the present invention

### 2) Mobile Health

In the present disclosure, “Mobile health” or “mHealth”, refers to the use of mobile devices such as smartphones, tablets, and wearables to deliver health services and information. The development of mHealth has been driven by advances in technology, increased access to mobile devices, and a growing demand for remote and personalized healthcare.

In the early days of mHealth, the focus was primarily on text messaging and voice calls to deliver health information and reminders to patients. Today, mHealth is being used in a variety of healthcare settings, including disease prevention, diagnosis, treatment, and management. It has been particularly useful in improving access to healthcare in remote and underserved areas, as well as in providing more personalized care to patients.

Some key benefits of mHealth include improved patient engagement, better health outcomes, and reduced healthcare costs. However, there are also challenges associated with mHealth, such as ensuring data privacy and security, addressing disparities in access to mobile technology, and integrating mHealth into existing healthcare systems. Overall, the development of mHealth has been a significant advancement in the field of healthcare.

### 3) Pose Estimation and Data Pre-Processing

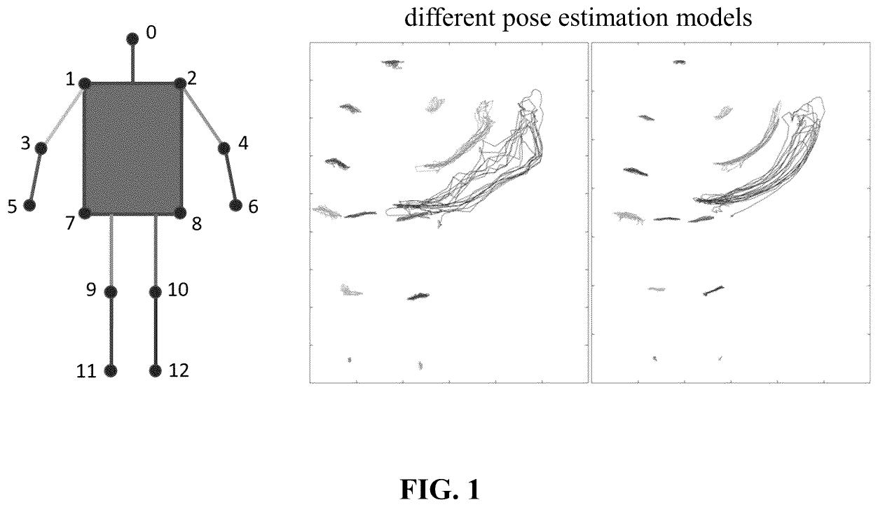

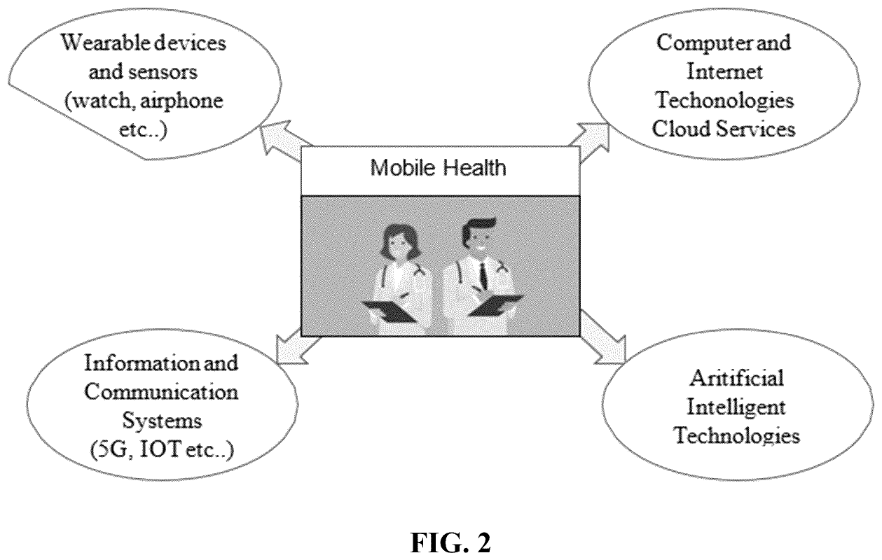

In some embodiment, pose estimation and data pre-processing are performed using software, such as MATLAB and Python. Two-dimensional (2D) Video recordings captured using the gold standard and consumer-grade devices are fed into at least one pre-trained model, such as the Kinect pre-trained pose estimation model and open source pre-trained MediaPipe pose estimation model. The x and y coordinates of the thirteen key joints are obtained and converted into time series data format for synchronizing the two sets of data, resulting in two blocks of 2*13 matrix based on the raw spatial location of the joints relative to resolution of capture device (see FIG. 1, there are 26 degrees of freedom). The time series data of the x and y coordinates of the key joints are passed through a high-pass filter using MATLAB signal processing toolbox to remove random spikes of key point coordinates from irrepresentable data frames. Notably, no smoothing technique is applied to the time series data, and the following data are processed in their raw format to represent the real-time and real-life usage of the pose estimation model for evaluating rehabilitation performance. After filtering and synchronizing the Kinect and MediaPipe skeleton data outputs, the video recordings are reviewed, and specific recording periods are manually labeled and segmented into their corresponding exercises to facilitate the validation analysis process, as shown in Table 1 as afore-mentioned.

Further, in the present disclosure, 2D pose estimation refers to the process of identifying the position and orientation of a human body in a 2D image or video. The development of 2D pose estimation technologies has been driven by advances in computer vision and machine learning algorithms, as well as improvements in hardware performance.

For algorithm development, in the early days of 2D pose estimation, simple algorithms are used to detect body parts based on colour and intensity. Over time, more sophisticated techniques were developed, including model-based approaches that used statistical models of human body shape and appearance to estimate pose. In some embodiments, deep learning methods such as convolutional neural networks (CNNs) are used for 2D pose estimation. These methods have achieved state-of-the-art performance on challenging datasets and can estimate pose in real-time on a wide range of devices.

### 4) Validation of Consumer-Grade Computer Vision Model Against Kinect Gold Standard

In the present disclosure, the performance of the predicted key joints using the open-source MediaPipe pose estimation model is compared against that of the gold standard Kinect pose estimation model. The Pearson correlation coefficient is used to examine each exercise's segmented time-series waveform similarity across the pose estimation models. To assess the ability of the MediaPipe pose estimation model to estimate and predict the end range of motion (ROM), three representative estimated end ROMs are manually selected out of the ten repetitions for each exercise. In some embodiments, estimated end ROM is computed using trigonometry properties (as Equation 1) while combining key joints into a vector (e.g., linking the wrist and elbow key joints as the forearm vector a, and elbow and shoulder key points as the proximal arm vector b to compute the elbow angle for elbow extension). The error between the end ROM prediction performance across the pose estimation models is analyzed using root-mean squared error (RMSE).

$$θ = {cos}^{- 1} ( \frac{a \cdot b}{\vert a \vert \vert b \vert} ) \qquad Equation 1$$

where, θ=angle of ∠ABC; a=vector from joint A to B; and b=vector from joint B to C.

In some embodiments, intraclass correlation (ICC) is performed using a mean-rating (k=2), absolute-agreement, 2-way mixed-effects model to analyze the agreement between the predicted end ROM across the pose estimation models. Values less than 0.5 are indicative of poor reliability, values between 0.5 and 0.75 indicate moderate reliability, values between 0.75 and 0.9 indicate good reliability, and values greater than 0.90 indicate excellent reliability. All statistical analyses are performed using MATLAB, and data are reported with a 95% confidence interval. A p-value <0.05 is considered statistically significant.

### 5) Sensors

In the present disclosure, sensor devices refer to devices that can detect and measure physical or chemical phenomena, such as temperature, pressure, light, sound, or motion. The development of sensor devices has been driven by advances in materials science, microelectronics, and wireless communication.

Simple sensors such as thermometers and pressure gauges are used to measure physical quantities. Over time, more sophisticated sensors are developed, including accelerometers, gyroscopes, and magnetometers, which are commonly used in smartphones and wearable devices. The miniaturization of sensors has also enabled the development of smart sensors, which are capable of processing data and making decisions based on that data. Besides, wireless communication technologies have also enabled the deployment of sensor networks, which are composed of multiple sensors that can communicate with each other and with a central server or cloud-based platform. Sensor networks can be used in a variety of applications, including smart homes, smart cities, and industrial automation.

In recent years, the development of sensors has been driven by the rise of the Internet of Things (IoT), which refers to the network of physical devices, vehicles, buildings, and other objects that are embedded with sensors, software, and connectivity. The IoT has enabled the integration of sensor data with other sources of data, such as social media, weather data, and traffic data, to create new insights and opportunities for innovation.

### 6) Development of a Telerehabilitation Model of Care

In view of the urgent need for innovative solutions to facilitate post-stroke rehabilitation services, a novel solution is provided to identify the limitations faced by current approaches that could potentially be addressed using the validated pose estimation model. A framework and sample solution implementing camera-based pose estimation technology alongside human activity evaluation methods for automated home-based stroke rehabilitation are developed.

Next, an architecture design of the present invention is provided as follows.

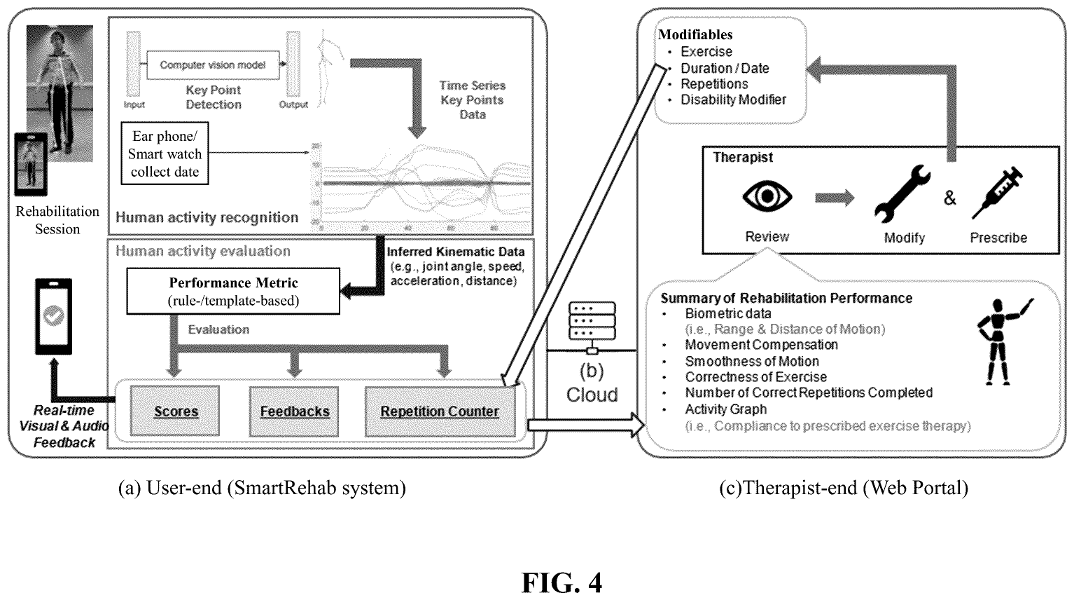

FIG. 4 depicts a schematic diagram outlining the framework of a system designed to facilitate telerehabilitation according to some embodiments of the present invention. In this regard, core technologies include human activity recognition (HAR) and human activity evaluation (HAE) on mobile devices, statistical methods, and web and database design and deployment.

The overview of the architecture design: a system is provided, called “SmartRehab system” and aiming to address the limitations faced by the current post-stroke rehabilitation methods and the avail-able literature and products mentioned above. The provided system incorporates interdisciplinary techniques and expert knowledge from Internet of Things (IoT), computer vision (CV), physiotherapy and neurology. In the illustration of FIG. 4, there are three sections. The system facilitates personalized and quality post-stroke rehabilitation through a cloud (see the part (b) of the illustration of FIG. 4) connecting the user-end (see the part (a) of the illustration of FIG. 4) and therapist-end (see the part (c) of the illustration of FIG. 4).

### The User-End Mobile Application of the Architecture Design:

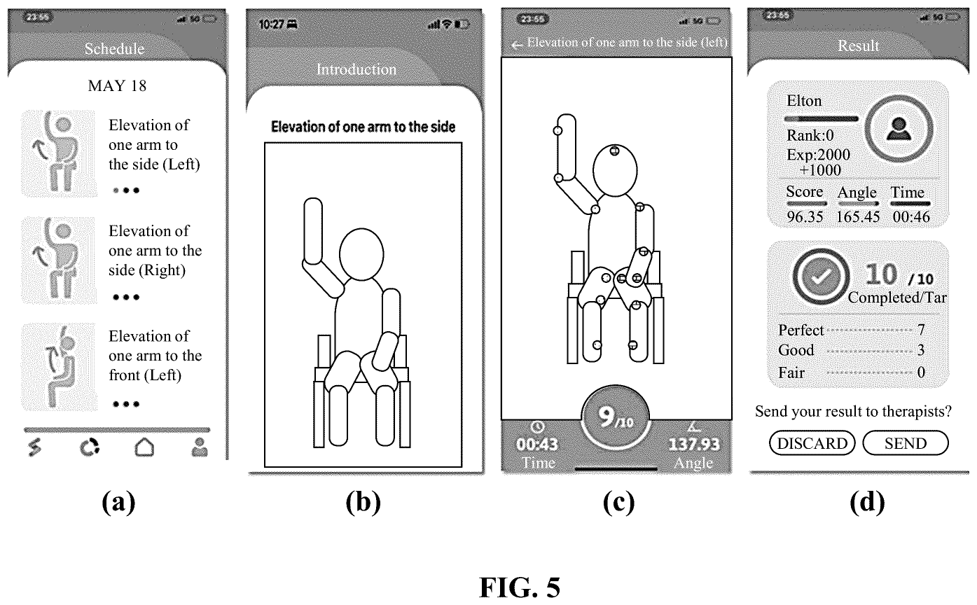

FIG. 5 depicts user interface of the mobile application for the system of FIG. 4 according to some embodiments of the present invention. There are four parts in the illustration of FIG. 5. Part (a) of the illustration of FIG. 5 is the homepage, which shows the schedule for the day. The Schedule page lists the active exercise plans prescribed by the therapist, such as various gross motor movements. Part (b) of the illustration of FIG. 5 is the previewing page, which displays video and written instructions for specific exercises. This instructions page includes both video demonstrations and written instructions on how to perform the exercises, provided by physiotherapists. Part (c) of the illustration of FIG. 5 is the camera view page, which interfaces to enable HAR and HAE to assess patients' rehabilitation quality. This camera page provides real-time pose estimation for repetition counting, range of motion estimation, exercise evaluation, and feedback functions, using AI and deep-learning technologies to track the user's movements. Part (d) of the illustration of FIG. 5 is the statistics page, which shows statistics and the progress of the patient's performance metrics. The results page provides a performance summary of the completed exercise session, with results recorded and sent back to the therapist.

More specifically, for user-end as shown in parts (a) and (b) of the illustration of FIG. 5, the patients can enter their rehabilitation exercise sessions as the physiotherapist prescribes, with specific targets being set (i.e., ten repetitions of left shoulder abduction per day). Using the provided human pose estimation (HPE) and activity evaluation module, the user-end allows automated evaluation of one's rehabilitation performance. As shown in the part (c) of the illustration of FIG. 5, the HPE model extracts relevant information from each camera input frame and predicts the key points of the human body. The provided HPE process can be done without additional markers or tracers needed. The human skeleton, biometrics and other movement data can then be inferred using the predicted key points.

In some embodiments, the HPE process utilizes the predicted human key points to infer relevant biometric data for analysis and evaluation against the gold standards determined by the experts from the Hong Kong Society for Rehabilitation (HKSR) and HKU Stroke.

For example, information such as joint angles, speed, acceleration, movement smoothness, key point distance, and more can be recorded. Using the inferred bodily data, the evaluation model can then accurately compare the time-series data against a gold-standard template or check joint angles against the gold-standard rules. These then are passed towards for scoring, feedback, and repetition counter functions. These summary data can then be used to provide real-time visual or audio feedback to the user, correcting and reinforcing quality rehabilitation performance, as shown in the part (d) of the illustration of FIG. 5. In one embodiment, gamification elements are also incorporated into the user interface to make the session more enjoyable and exciting, motivating and facilitating rehabilitation exercises at home.

The Therapist-end Web Portal System of the architecture design:

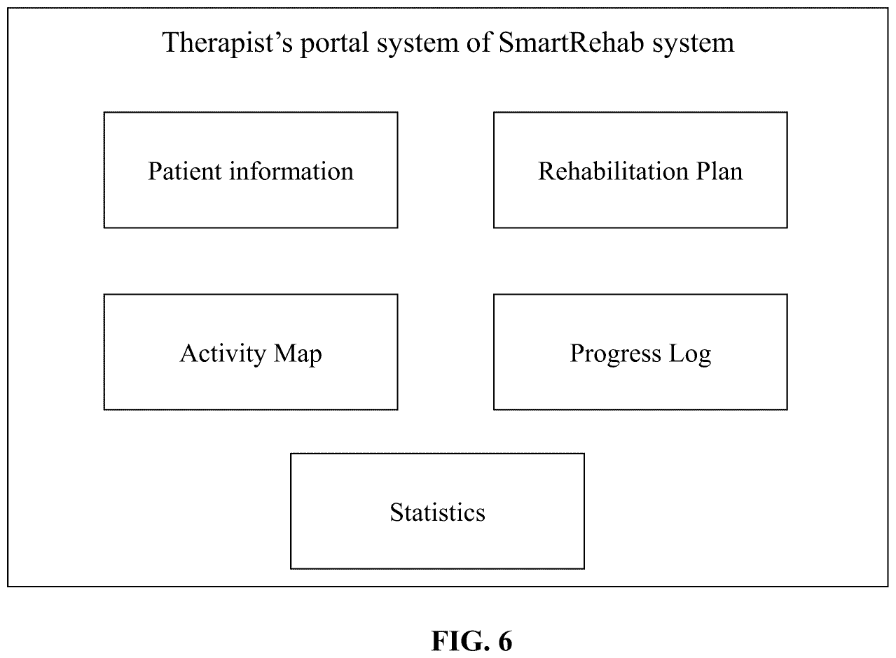

FIG. 6 depicts a schematic diagram for showing a therapist's portal system of the SmartRehab system of FIG. 4 for objectively reviewing patient's exercise performance according to some embodiments of the present invention. The portal system includes function of “Patient information,” “Activity Map,” “Rehabilitation Plan,” “Progress Log,” and “Statistics.” These functions can be reviewed as website pages or via app (e.g., smart phone operation).

Patient information is for providing a brief summary of the patient's condition and rehabilitation goal.

Activity Map is for visualizing daily compliance and completeness of the prescribed exercises using a traffic light system; for example, it can be defaulted as: Red=Incomplete, Yellow=Partially complete, and Green=Completed).

Rehabilitation Plan is as a calendar format for showing actively prescribed exercise plan and prescription of new plans.

Progress Log is for logging therapist actions on the patient profile, including edits in patient information or prescription or modification to exercise plan, etc.

Statistics is for reviewing exercise performance as measured and estimated by the pose-estimation model: Range of motion: average angle or distance of interest achieved during home-based exercise (e.g., angle between shoulder and the elbow from vertical for shoulder abduction); Quality of performance: detection of trick movements (e.g., which are as identified by the HKSR); Count: repetitions; Movement trajectory: for visualization and qualitative analysis of the key exercise waveform (e.g., angle between shoulder and the elbow from vertical for shoulder abduction).

The Therapist-end Web Portal System of the architecture design:

Once the users have completed their rehabilitation sessions, they can upload their rehabilitation performance data onto the cloud service via a cloud path as shown in part (b) of the illustration of FIG. 4).

Therapists and healthcare professionals remotely access the patient's performance data by logging into the password-protected and encrypted portal system, as shown in part (c) of the illustration of FIG. 4 and the illustration of FIG. 6). The web portal system is designed for therapists to remotely navigate and monitor patients' performance and progress of their condition. The portal system includes a summary of patients' details (e.g., patient particulars, active illness, rehabilitation goals, and relevant past medical history).

In various embodiments, the web portal system may have an activity map adopting a traffic light report system to visualize the activity of patients on their assigned or prescribed exercises; for example: a “green light” signifies the completion of all prescribed exercises; a “yellow light” for partial completion; and a “red light” for incomplete. The activity map serves to monitor one's compliance and potential for identifying patients who may be experiencing difficulty with the platform. The progress log logs the therapist's actions on the patient profile, including edits in patient information or prescription or modification to the exercise plan, etc.

In various embodiments, the web portal system may include a statistic panel that can showcase three primary data domains that are of significant interest in facilitating telerehabilitation (e.g., domains which are identified by the Hong Kong Society for Rehabilitation (HKSR) experts). These domains include the “range of motion” of the joints of interest for the particular exercise, the “quality of exercise” when compared to the experts' criteria and metrics, and “repetition counts.” Therapists can regularly monitor and follow up on their patients' performance and compliance through the web portal, and further adjust their exercise difficulty and intensity to facilitate a seamless telerehabilitation process. Meanwhile, as the patient has achieved adequate performance as reflected by the scores from the scoring function, the therapist can proceed to modify the plan and increase the difficulty to further challenge the user for further improvement. Through the portal system, therapists can easily modify the modifiable, including the type of exercise, the number of target repetitions, the duration or frequency of the exercise, and the target range of motion can all be customized towards one's need. Altogether, these modifiers offer excellent flexibility for allowing a personalized rehabilitation plan for fitting patients' needs.

Accordingly, the SmartRehab system includes two unique interfaces connected via a cloud server-a mobile application for stroke patients in need of rehabilitation services (i.e., the user-end) and a web-based portal system for therapists and healthcare professionals to manage their patients (i.e., the therapist-end).

In response to the provided system, Results and Evaluation is provided as follows.

The SmartRehab system can encompass ten gross movement exercises, six of which involve the upper limb, as shown in Table 2 below.

TABLE 2
List of exercises currently available within SmartRehab system
Exercises in the SmartRehab system

Unilateral/Bilateral shoulder abduction*
Unilateral/Bilateral shoulder flexion*
Hemiplegic assisted shoulder flexion*
Unilateral elbow extension*
Hemiplegic assisted elbow extension*
Bilateral shoulder external rotation
Single leg stance
Pre-gait swing
Standing weight shift
Sitting weight shift
*means upper limb exercises

These exercises are recommended and designed by therapists from the HKSR and are specifically tailored for stroke patients to improve upper limb function, weight shifts, and balance. In contrast to traditional telerehabilitation platforms such as Kinect, the SmartRehab system utilizes the built-in RGB camera of the tablet or smartphone device. Correspondingly, a computer vision-based pose estimation algorithm, which uses a deep learning model to predict precise human key points and segment key joint locations, is developed and provided herein.

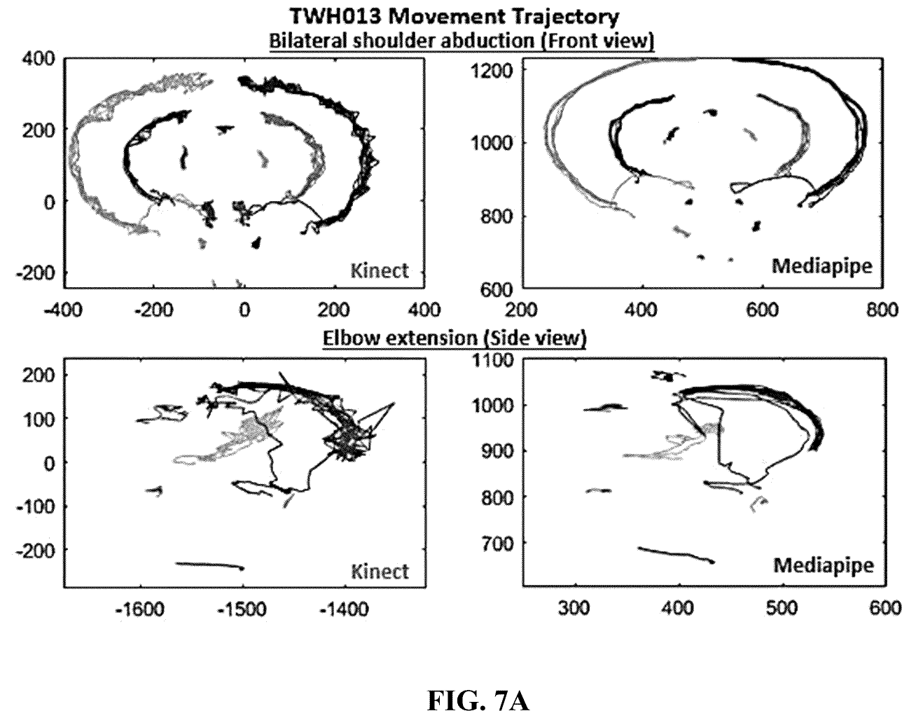

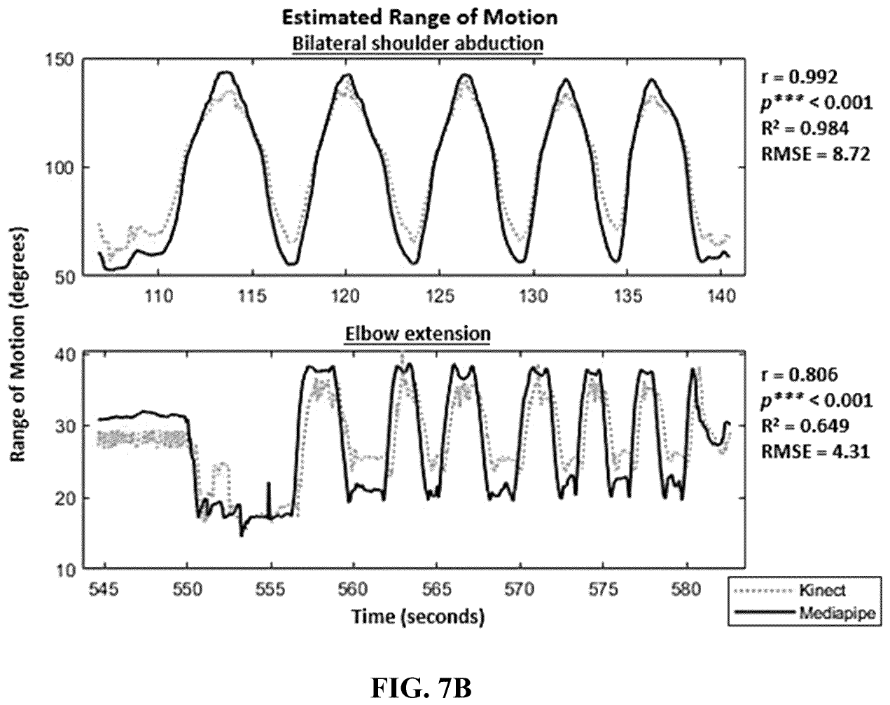

FIG. 7A and FIG. 7B show graphs for visualization of movement waveforms captured during shoulder abduction and elbow extension. Waveform data can be extracted for computing angles (x vs y vs z in space), angular velocity (θvs time), and spasticity (smoothness of curve). The provided system allows SmartRehab to compute movement-related features, including movement trajectory (see FIG. 7A), changes in joint angles, velocity, and the presence of spastic movement (see FIG. 7B), all of which can be used to evaluate the quality of patients' movement and provide immediate feedback.

Besides, as mentioned in the introduction, Kinect is a markerless motion capture device widely implemented in verified and center-based settings for rehabilitation. However, its cost, computational requirements, and need for specific setup limit its accessibility and wide dissemination for home-based telerehabilitation. Therefore, for better evaluation, the pose estimation model of the SmartRehab system is validated against Kinect. These data points are in a range of residual upper limb impairments (44% with Medical Research Council power of proximal upper limb<5; 44% with Modified Ashworth Scale of proximal upper limb<0). The results demonstrate that the movement trajectory and range of motion are similar between the SmartRehab system and Kinect (see FIG. 7A). In addition, the intraclass correlations between the predicted range of motion of the upper limb detected (abduction, shoulder flexion, and elbow extension) using the SmartRehab system and Kinect are all close to one, as shown in Table 3 below. The excellent results further manifest the promise of automated telerehabilitation and prescription-based platforms.

TABLE 3
Root-mean squared error (RMSE) and intraclass correlation
coefficient analysis of an active range of motion prediction
between Kinect and the SmartRehab system.

| | Root-mean | | |
|---|---|---|---|
| | squared error | | |
| Exercise | (degrees) | Intraclass correlation | |
| Shoulder abduction | 9.65 | 0.977 | (0.965-0.985) |
| Shoulder flexion | 9.268 | 0.971 | (0.956-0.981) |
| Elbow extension | 2.986 | 0.915 | (0.863-0.948) |

More results are provided below. Several data points are collected in the sensor validation study, as shown in Table 4 below.

TABLE 4
Demographics and characteristics of stroke
patients in the validation study.
Demographics (N = 9)

| Median Age in years (IQR) | 60 | (56-67) |
|---|---|---|
| Gender; n (%) | Male: n = 7 (78%) | |
| Median Height in cm (IQR) | 169 | (165-174) |
| Median Weight in kg (IQR) | 67 | (63-70) |

Medical Record

| Median Days since hospital | 97 | (65-240) |
|---|---|---|
| admission when assessed (IQR) | | |
| Stroke type; n (%) | Hemorrhagic: n = 6 (67%) | |
| | Ischemic: n = 3 (33%) | |
| Prior stroke; n (%) | 0 | (0%) |

| | |
|---|---|
| Neurological | Yes: n = 1 (11%) |
| conditions; n (%) | |
| Walking aids; n (%) | Walking stick: n = 3 (33%) |
| | Wheelchair: n = 1 (11%) |

Clinical Score

| | |
|---|---|
| Medical Research Council | MRC 5: n = 5 (56%) |
| (MRC) of Proximal upper | MRC 3: n = 2 (22%) |
| limb; n (%) | MRC 2: n = 2 (22%) |
| Modified Ashworth Scale | MAS 0: n = 5 (56%) |
| (MAS) of Proximal upper | MAS 1: n = 3 (33%) |
| limb; n (%) | MAS 3: n = 1 (11%) |
| Modified Rankin Scale | mRS < 3: n = 6 (67%) |
| (mRS); n (%) | mRS ≥ 3: n = 3 (33%) |

Of the nine data points (i.e., nine participants) that proceeded to data pre-processing and validation analysis, seven of them are male. The participants have an overall median age of 60 years (IQR: 56-67 years), height of 169 cm (IQR: 165-174 cm), and weight of 67 kg (IQR: 63-70 kg). Median days since hospital admission when assessed and video captured was 97 days (IQR: 65-240). Six participants experienced hemorrhagic stroke, and three with ischemic stroke. None of them had experienced a prior stroke. One participant has paranoid-type schizophrenia but with minimal motor manifestation. Four participants require walking aids, three of which require a walking stick, and one requires a wheelchair. Regarding motor performance at the time of assessment and video capture, five participants had full Medical Research Council (MRC) score over their proximal upper limb of the affected side, two with MRC score of 3, and two with MRC score of 2. Muscle tone assessment using Modified Ashworth Scale (MAS) result showed five participants with MAS of 0, three with MAS of 1, and one with MAS of 3. Overall functional disability assessment using Modified Rankin Scale (mRS) showed six participants had no symptoms to mild disability (mRS<3), and three with moderate to severe disability (mRS≥3) after stroke.

### Regarding Validation Analysis:

A perfect correlation is found between the waveform movement across all upper limb exercises in all participants when comparing the pose estimation performance of MediaPipe against Kinect, as shown in Table 5 below.

TABLE 5
Pearson correlation of movement trajectory between pose estimation
models MediaPipe and Kinect during upper limb exercises.

| | Pearson correlation | |
|---|---|---|
| Movement | (95% CI) | P-value |
| Shoulder Abduction | 0.98 (0.97-0.99) | <.001 |
| Shoulder Flexion | 0.97 (0.96-0.98) | <.001 |
| Elbow Extension | 0.87 (0.80-0.92) | <.001 |

Notably, a significantly smoother movement trajectory and range of motion waveform is observed in the MediaPipe pose estimation output without applying any smoothing filter or processing to the data, whereas the Kinect pose estimation output appears more “jagged” (e.g., FIG. 1, FIG. 7A, and FIG. 7B). The “jagged” appearance can be attributed to the signal noises captured by the infrared camera.

RMSE analysis of differences in end ROM between MediaPipe and Kinect output for shoulder abduction, shoulder flexion, and elbow extension is 9.65, 9.27, and 2.99 degrees, respectively, as shown in Table 6 below. ICC analysis shows significant and excellent reliability in the MediaPipe pose estimation model for predicting end ROM in all exercises compared to the Kinect pose estimation model. ICC results are 0.98 (95% CI: 0.96-0.99), 0.97 (0.80-0.99), and 0.92 (0.86-0.95) for the above exercises, respectively.

TABLE 6
Root-mean squared error (RMSE) and Intraclass correlation (ICC)
analysis of end range of motion (ROM) prediction between MediaPipe
and Kinect pose estimation models during upper limb exercises.

| | RMSE | ICC of end ROM | |
|---|---|---|---|
| Movement | (Degrees) | (95% CI) | P-value |
| Shoulder Abduction | 9.65 | 0.98 (0.96-0.99) | <.001 |
| Shoulder Flexion | 9.27 | 0.97 (0.80-0.99) | <.001 |
| Elbow Extension | 2.99 | 0.92 (0.86-0.95) | <.001 |

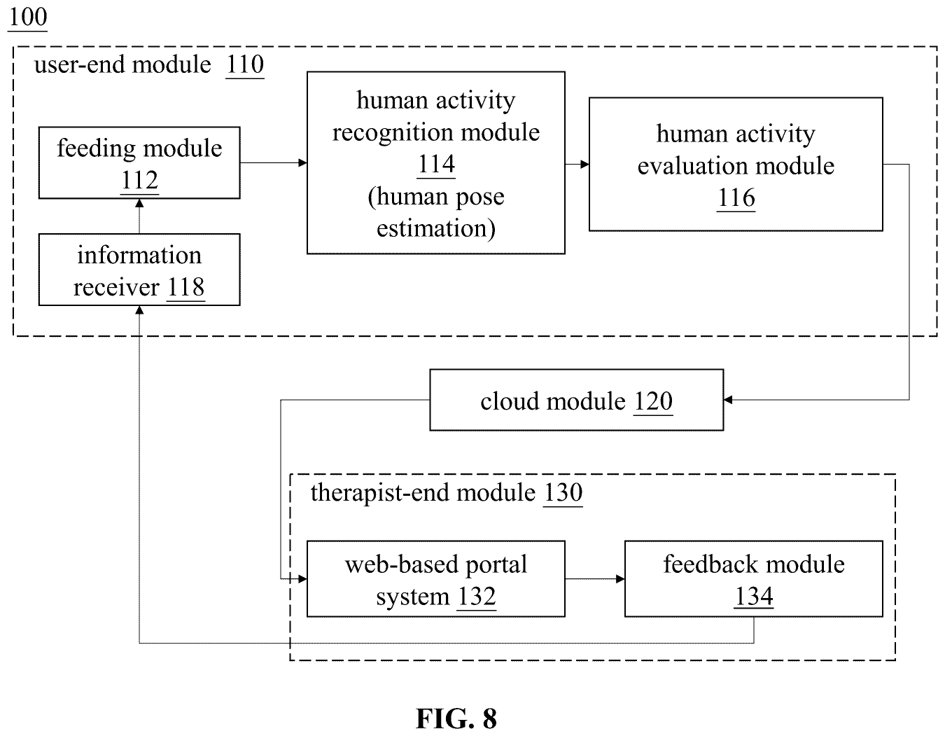

FIG. 8 depicts a schematic architecture of a system 100 for a personalized community-based post-stroke rehabilitation using SmartRehab according to one embodiment of the present invention. The system 100 includes a user-end module 110, a cloud platform module 120, and a therapist-end module 130 in communication with the user-end module 110 via the cloud platform module 120. The user-end module 110 is configured to provide schedule information with instructions of at least one specific exercise and show at least one image or video, in which the user-end module 110 can provide a camera view page to record a target image or video by a camera and record a target user's performance metric. The cloud platform module 120 is configured to receive and store the target image or video and the target user's performance metric from the user-end module 110. The therapist-end module 130 permitted to log in the cloud platform module 120 and receive the target image or video and the target user's performance metric from the cloud platform module 120, in which the therapist-end module 130 is further configured to visualize the target user's performance metric so as to show exercise waveform including quantitative data for qualitative analysis. In one embodiment, the target user's performance metric comprises joint angles, speed, acceleration, movement smoothness, key point distance, and combinations thereof.

More specific details are provided as below.

The user-end module 110 includes a feeding module 112, a human activity recognition module 114, a human activity evaluation module 116, and an information receiver 118. The user-end module 110 is capable of being installed on a smartphone.

The feeding module 112 is configured to acquire photos or videos (e.g., RGB-digital camera input) from a consumer-grade smartphone or tablet device, which serve as the primary data sources. Once the photos or videos are obtained, they are input into the human activity recognition module 114. The human activity recognition module 114 leverages a human pose estimation model, implemented using convolutional neural networks (CNNs), to analyze the input data. The human pose estimation model identifies and predicts the position and orientation of various body joints, providing detailed information about the participant's movements.

For example, the human pose estimation model extracts relevant information from each camera input frame and predicts the key points of the human body. This process can be done without additional markers or tracers. The human skeleton, biometrics, and other movement data can then be inferred using the predicted key points. In some embodiments, the human pose estimation process utilizes the predicted human key points to infer relevant biometric data for analysis and evaluation. For example, information applied by the human pose estimation model includes joint angles, speed, acceleration, movement smoothness, and key point distance.

In various embodiments, the human pose estimation model includes computer vision models able to predict the human body and key point locations in space (e.g., the x and y coordinates of the elbow joint in space at time t). The performance is then objectively and automatically evaluated with the inference of joint angles, velocity, smoothness of movement, and other properties using the body frame key points. The human pose estimation process can use a deep learning model to predict precise human key points and segment key joint locations.

Following the analysis, the results are transmitted to the human activity evaluation module 116. The human activity evaluation module 116 evaluates the performance based on performance metric information, which can be rule-based or template-based. The evaluation results from the human activity evaluation module 116 can be transmitted to the therapist-end module 130 through the cloud platform module 120.

In this regard, various rehabilitation exercises require attention to specific evaluation angles. By focusing on the angles formed by three crucial points associated with each exercise and referencing the thresholds set by the Hong Kong Society for Rehabilitation (HKSR), patient rehabilitation exercises can be effectively evaluated. For example, as considered the shoulder abduction exercise, which focuses on two angles: the left elbow-left shoulder-left hip and the right elbow-right shoulder-right hip.

It is assumed that the coordinates of the right elbow point are obtained as (x₁, y₁); the coordinate of the right shoulder point are obtained as (x₂, y₂); and the coordinate of the right hip point are obtained as (x₃, y₃). Then, there are two vectors are determined:

$${V}_{1} = ( ( {x}_{1} - {x}_{2} ) , ( {y}_{1} - {y}_{2} ) )$$

$${V}_{2} = ( ( {x}_{3} - {x}_{2} ) , ( {y}_{3} - {y}_{2} ) )$$

Next, the key point angle can be calculated as:

$$θ = c o {s}^{- 1} [ ( {V}_{1} \cdot {V}_{2} ) / ( \vert {V}_{1} \vert \vert {V}_{2} \vert ) ]$$

$$where \{ \begin{matrix}Perfect , 150 ° < θ < 180 ° \\ Good , 90 ° < θ < 150 ° \\ Fair , 45 ° < θ < 90 °\end{matrix}$$

The average angle and score are computed as the final assessment outcome for each batch of exercises, where each batch consists of n repetitions. This evaluation method enables the provided SmartRehab system to transmit patient assessment data from their portable device to the rehabilitation specialist's system. As a result, the therapist can easily monitor the patient's recovery progress and make real-time adjustments as needed.

In practical application settings, patients may perform inaccurate movements due to the absence of direct oversight from their therapist. These incorrect actions can lead to inefficient rehabilitation exercises or even negatively impact the rehabilitation process. To address this challenge, the SmartRehab system employs a reference angle for routine evaluation of rehabilitation movements and considers potential errors by providing textual and visual guidance. For instance, in the Shoulder Abduction exercise, the standard assessment angle is the left elbow-left shoulder-left hip angle on the X-Y plane. Conversely, the evaluation of incorrect movements is based on the left elbow-left shoulder-right shoulder angle on the X-Z plane. This approach helps in identifying and correcting any inaccuracies during the exercise.

The therapist-end module 130 includes a web-based portal system 132 and a feedback module 134. The therapist-end module 130 is permitted to log in the cloud platform module 120 and send a session instruction to be stored in the cloud platform module 120, which is sent to the user-end module 110 and is permitted to amend the schedule information and the video and written instructions of the user-end module 110. The web-based portal system 132 allows healthcare professionals to manage a patient's profile remotely, ensuring that therapist-instruction information is updated as needed. Based on the updated therapist-instruction information stored in the web-based portal system 132, the feedback module 134 can send signals to the information receiver 118 of the user-end module 110, enabling the user to undergo the next evaluation cycle.

To summarize the aforementioned, the user-end feeds the RGB-digital camera input from a consumer-grade smartphone or tablet device into two modules, namely the human activity recognition with human pose estimation, followed by the human activity evaluation module. We employed the MediaPipe human pose estimation model validated above and fed the key joints information into the rule-based human activity evaluation model, where performances of the enlisted exercises can be evaluated according to the pre-determined criteria and metrics developed by experienced therapists from the HKSR. Qualitative analysis of the exercise can be done using kinematic features inferred from the waveform movement of the key joints, including the location of joints (demonstrating the baseline pose, e.g., flexor tone), distance, velocity, and acceleration of the movement, presence of spastic jerky movement, and end range of motion. Quantitative analysis can be performed by detecting the number of repetitions and sessions completed. Real-time feedback can be provided using the key joints data to inform users of their correctness and provide reminders through pre-determined and handcrafted rules. Furthermore, the detection, evaluation, and feedback process were automated using objective measures (i.e., key points and inferred qualitative and quantitative data), re-enacting a therapist monitoring one's performance, altogether reducing the workload required as compared with an in-person service.

Then, the web-based portal system on the therapist-end allows healthcare professionals to manage one's profile remotely. Qualitative and quantitative objective measurements from the automated human activity recognition and human activity evaluation modules on the user-end mobile application were collected and uploaded onto the cloud server. Therapists can access, and prescribe new exercise plans, review progress and performance, and modify current exercise plans. Therapists have high flexibility in modifying and customizing exercise plans, where properties can be personalized to the patient's need, including the number of sessions, repetition per day, and the difficulty of the exercise. Completeness of the prescribed exercise plan can easily be quantified and tracked using a traffic light report for monitoring adherence and compliance. Overall, our framework for telerehabilitation enables the remote distribution and dissemination of services to areas with adequate internet services through more readily available consumer-grade smartphone or tablet devices, without additional specialized sensors or equipment.

As discussed above, the rule-based human activity evaluation module employed in SmartRehab utilizes objective measures through the predicted and inferred kinematic data. Currently available telemedicine and telerehabilitation solutions rely primarily on one's subjective recall, leading to biased responses. By using key joint markers that are accurate and reliable, automated continuous assessment and monitoring of rehabilitation exercise performance is enabled, which could be labor-intensive and nearly impossible in in-person services. Important kinematic information, including body posture, velocity, acceleration, and spasticity of movement, can be easily assessed without additional work. However, rule-based evaluation modules require expert opinions and handcrafted features, which may be difficult or near-impossible in relatively complicated and complex exercises. Moreover, the rule-based evaluation algorithm's accuracy, reliability, and effectiveness depend on the quality and comprehensiveness of the pre-determined criteria (e.g., the expected end ROM in a healthy subject, any trick movements that may resemble the exercise, any specific instructions or warnings). Future directions may incorporate deeper classification models and utilize template-based or other evaluation approaches with higher dimensionality to further automate the evaluation process.

SmartRehab leverages AI and computer vision for automated posture analysis and performance evaluation during home-based stroke rehabilitation exercises. The app acts as a “digital coach” to enable patients to continue rehabilitation remotely in the community. Therapists can prescribe personalized exercise plans through the SmartRehab portal and monitor progress. Physiological metrics collected during exercises provide valuable data to inform treatment. This platform potentially reduces the need for face-to-face sessions with a therapist, reduces travel time to rehabilitation centers, and is particularly valuable during situations such as COVID-19, where social distancing is needed to minimize cross-infection in rehabilitation centers.

The functional units and modules of the apparatuses and methods in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes executing in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can be included, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A system for providing personalized community-based post-stroke rehabilitation, comprising:
a user-end module configured to provide schedule information with instructions of at least one specific exercise and to show at least one image or video, wherein the user-end module provides a camera view page to record a target image or video and to record a target user's performance metric, wherein the user-end module comprises:
a feeding module configured to acquire the image or video from a consumer-grade smartphone or tablet device;
a human activity recognition module configured to receive the image or video from the feeding module and to leverages a human pose estimation model to analyze input data; and
a human activity evaluation module configured to receive results transmitted from the human activity recognition module and to evaluate performance of the results based on performance metric information which is rule-based or template-based;
a cloud platform module configured to receive and store the target user's performance metric from the user-end module; and
a therapist-end module in communication with the user-end module via the cloud platform module, wherein the therapist-end module is permitted to log in the cloud platform module and receive the target user's performance metric from the cloud platform module, and wherein the therapist-end module is further configured to visualize the target user's performance metric so as to show exercise waveform comprising quantitative data for qualitative analysis.

2. The system according to claim 1, wherein the cloud platform module is further configured to store the target image or video from the user-end module, and wherein the therapist-end module is permitted to receive and store the target image or video from the cloud platform module.

3. The system according to claim 1, wherein the target user's performance metric comprises joint angles, speed, acceleration, movement smoothness, key point distance, and combinations thereof.

4. The system according to claim 1, wherein the human pose estimation model of the human activity recognition module is implemented using convolutional neural networks (CNNs) to analyze the input data, and wherein the human pose estimation model is configured to identify and predict a position and orientation of various body joints according to the input data, providing detailed information about user's movements.

5. The system according to claim 4, wherein the human pose estimation model is configured to extract relevant information from each input frame of the target image or video and to predict key points of a body appearing in the target image or video.

6. The system according to claim 5, wherein the human pose estimation model is configured to use the predicted key points to infer skeleton, biometrics, and movement data.

7. The system according to claim 6, wherein the human pose estimation model is configured to conduct a human pose estimation process that utilizes the predicted key points to infer relevant biometric data for analysis and evaluation, and information applied to the human pose estimation process by the model includes joint angles, speed, acceleration, movement smoothness, and key point distances.

8. The system according to claim 7, wherein the human pose estimation model comprises computer vision models for predicting the body and key point locations in space as two-dimensional coordinates at a given time.

9. The system according to claim 8, wherein the human activity evaluation module is further configured to transmit evaluation results to the therapist-end module through the cloud platform module, and wherein the therapist-end module comprises:
a web-based portal system configured to allow an external accessor to manage a user's profile remotely according to the evaluation results, ensuring that therapist-instruction information is updated for the user-end module.

10. The system according to claim 9, wherein the therapist-end module further comprises:
a feedback module in communication with the web-based portal system, wherein the feedback module is configured to send a session instruction to the user-end module and is permitted to amend the schedule information and the instructions of the specific exercise of the user-end module.

11. A method for providing personalized community-based post-stroke rehabilitation, comprising:
providing, by a user-end module, schedule information with instructions of at least one specific exercise;
showing, by the user-end module, at least one image or video, wherein the user-end module provides a camera view page to record a target image or video and to record a target user's performance metric, wherein the user-end module comprises:
a feeding module configured to acquire the image or video from a consumer-grade smartphone or tablet device;
a human activity recognition module configured to receive the image or video from the feeding module and to leverages a human pose estimation model to analyze input data; and
a human activity evaluation module configured to receive results transmitted from the human activity recognition module and to evaluate performance of the results based on performance metric information which is rule-based or template-based;
receiving and storing, by a cloud platform module, the target user's performance metric from the user-end module;
logging, by using a therapist-end module, in the cloud platform module to receive the target user's performance metric from the cloud platform module; and
visualizing, by using the therapist-end module, the target user's performance metric so as to show exercise waveform comprising quantitative data for qualitative analysis.

12. The method according to claim 11, further comprising:
storing, by the cloud platform module, the target image or video from the user-end module, wherein the therapist-end module is permitted to receive and store the target image or video from the cloud platform module.

13. The method according to claim 11, wherein the target user's performance metric comprises joint angles, speed, acceleration, movement smoothness, key point distance, and combinations thereof.

14. The method according to claim 11, wherein the human pose estimation model of the human activity recognition module is implemented using convolutional neural networks (CNNs) to analyze the input data, and wherein the human pose estimation model is configured to identify and predict a position and orientation of various body joints according to the input data, providing detailed information about user's movements.

15. The method according to claim 14, further comprising:
extracting, by the human pose estimation model, relevant information from each input frame of the target image or video; and
predicting, by the human pose estimation model, key points of a body appearing in the target image or video.

16. The method according to claim 15, wherein the human pose estimation model is configured to use the predicted key points to infer skeleton, biometrics, and movement data.

17. The method according to claim 16, further comprising:
conducting, by the human pose estimation model, a human pose estimation process that utilizes the predicted key points to infer relevant biometric data for analysis and evaluation, wherein information applied to the human pose estimation process by the model includes joint angles, speed, acceleration, movement smoothness, and key point distances.

18. The method according to claim 17, wherein the human pose estimation model comprises computer vision models for predicting the body and key point locations in space as two-dimensional coordinates at a given time.

19. The method according to claim 18, further comprising:
transmitting, by the human activity evaluation module, evaluation results to the therapist-end module through the cloud platform module; and
allowing, by using a web-based portal system of the therapist-end module, an external accessor to manage a user's profile remotely according to the evaluation results, thereby ensuring that therapist-instruction information is updated for the user-end module.

20. The method according to claim 19, further comprising:
sending, by using a feedback module of the therapist-end module, a session instruction to the user-end module so as to amend the schedule information and the instructions of the specific exercise of the user-end module.
